(19) United States
(12) Patent Application Publication
(10) Pub. No.: US 2025/0387127 A1
(43) Pub. Date: Dec. 25, 2025

(54) BIONIC MICROTEXTURE MICRO GRINDING HEAD AND PROCESSING PROCESS THEREOF

(71) Applicants: QINGDAO UNIVERSITY OF TECHNOLOGY, Shandong (CN); QINGDAO JIMO QINGLI INTELLIGENT MANUFACTURING INDUSTRY RESEARCH INSTITUTE, Shandong (CN); GUOHUA (QINGDAO) INTELLIGENT PRECISION DRIVE CONTROL TECHNOLOGY RESEARCH INSTITUTE CO., LTD., Shandong (CN)

(72) Inventors: Min Yang, Shandong (CN); Zhonghao Li, Shandong (CN); Jiachao Hao, Shandong (CN); Guang Wang, Shandong (CN); Yongxi Cheng, Shandong (CN); Xiaoming Wang, Shandong (CN); Zongming Zhou, Shandong (CN); Changhe Li, Shandong (CN); Yanbin Zhang, Shandong (CN); Xiao Ma, Shandong (CN); Benkai Li, Shandong (CN); Mingzheng Liu, Shandong (CN); Xin Cui, Shandong (CN); Teng Gao, Shandong (CN)

(73) Assignees: QINGDAO UNIVERSITY OF TECHNOLOGY, Shandong (CN); QINGDAO JIMO QINGLI INTELLIGENT MANUFACTURING INDUSTRY RESEARCH INSTITUTE, Shandong (CN); GUOHUA (QINGDAO) INTELLIGENT PRECISION DRIVE CONTROL TECHNOLOGY RESEARCH INSTITUTE CO., LTD., Shandong (CN)

(21) Appl. No.: 18/964,640

(22) Filed: Dec. 1, 2024

(30) Foreign Application Priority Data
Jun. 24, 2024 (CN) 202410819506.9

(51) Int. Cl. A61B 17/16; A61B 17/00

(52) U.S. Cl. CPC A61B 17/1615; A61B 2017/00526; A61B 2017/1602

(57) ABSTRACT

A bionic microtexture micro grinding head includes a micro grinding head base, the micro grinding head base includes a connecting member, the connecting member is connected to a grinding head, grain groups or grains are sequentially disposed on a surface of the grinding head, the grain groups are arranged according to a bionic pinion microstructure or a bionic scale structure, and the grains are arranged according to a bionic phyllotaxis microstructure. When the grain groups are arranged according to the bionic pinion microstructure or the bionic scale structure, each grain group includes grains; or, when the grain groups are arranged according to the bionic pinion microstructure or arranged according to the bionic scale structure, a groove is provided between two adjacent grain groups.

### CROSS-REFERENCE TO RELATED APPLICATION

This application claims the priority benefit of CN application No. 202410819506.9, filed on Jun. 24, 2024. The entirety of each of the above-mentioned patent applications is hereby incorporated by reference herein and made a part of this specification.

### TECHNICAL FIELD

The present invention relates to the field of bone grinding technologies, and in particular, to a bionic microtexture micro grinding head and a processing process thereof.

### BACKGROUND

The statements in this section merely provide background information related to the present invention, and do not necessarily constitute the prior art.

Bone grinding is one of common and basic surgical operations in surgery for removing tumors from the base of the skull. Surgeons often remove bone pathology by using high-speed micro grinding tools such as grinding heads rather than grinding wheels in the clinic. However, high-speed grinding generates a large amount of heat, resulting in osteonecrosis and thermal damage to surrounding tissue, and also having a particular impact on a blood clotting function of tissue. Drop cooling of physiological saline is commonly used in the clinic, and part of grinding heat is taken away through natural convection. Grinding thermal damage is of recognized clinical concern because a temperature cannot be determined in a grinding process and a degree of thermal damage cannot be controlled. Researchers point out that a maximum critical temperature of thermal damage in a pouring physiological saline cooling manner is 43° C. At a temperature above 43° C., the optic nerve is subject to damage, and blindness is caused in severe cases. Regarding bone grinding, facial paralysis and femoral head necrosis are also common problems in orthopedic surgery. Therefore, in bone grinding surgery, the control of the temperature is directly related to the success of the surgery.

The inventor found that in an existing grinding head, due to an inappropriate arrangement of grains, the grains agglomerate during electroplating, resulting in a concentrated grinding stress in a grinding process, and large thermal-mechanical damage in a bone grinding process affects the success of the surgery. In addition, the inappropriate arrangement of grains affects a material removal rate in grinding, resulting in an increase in surface roughness of an object to be ground.

In addition, the grains on the surface of the grinding head are prone to wear, and the grains are prone to microfracture and falling off. In the prior art, although a bionic structure is used for a grinding wheel to improve the structure of the surface of the grinding wheel to improve the efficiency of cooling that the surface of the grinding wheel joins in a grinding process of the grinding wheel, for a specific bionic structure used to be applied to a grinding head with grains, there is no research in related directions.

### SUMMARY

In view of the deficiencies existing in the prior art, an objective of the present invention is to provide a bionic microtexture micro grinding head, to improve grinding efficiency and improve processing quality of a surface of a processing object.

To achieve the foregoing objective, the present invention is implemented by using the following technical solutions:

A bionic microtexture micro grinding head includes a micro grinding head base. The micro grinding head base includes a connecting member, the connecting member is connected to a grinding head, a plurality of grain groups or a plurality of grains are sequentially disposed on a surface of the grinding head, the plurality of grain groups are arranged according to a bionic pinion microstructure or a bionic scale structure, and the plurality of grains are arranged according to a bionic phyllotaxis microstructure;
when the plurality of grain groups are arranged according to the bionic pinion microstructure or the bionic scale structure, each grain group includes a plurality of grains, and when the plurality of grain groups are arranged according to the bionic pinion microstructure or arranged according to the bionic scale structure, a groove is provided between two adjacent grain groups;
when the plurality of grains are arranged according to the bionic phyllotaxis microstructure, the plurality of grains are disposed in a plurality of rows and a plurality of columns, rows in which some grains are located and columns in which some grains are located are respectively disposed obliquely with respect to a center-crossing radial plane of the grinding head, and the center-crossing radial plane is disposed perpendicular to a central axis of the connecting member; and
the grains are disposed protruding from the surface of the grinding head.

For the micro grinding head described above, the grain groups or the grains are disposed on the surface of the grinding head. The grain groups or the grains are arranged according to various bionic structures, the layout of grains of a conventional grinding head is changed, and the grains are arranged in order, so that concentration of a grinding force is reduced, and thermal-mechanical damage in a bone grinding process is reduced, thereby avoiding a surgical problem caused by a high temperature. The improved micro grinding head can effectively improve the grinding efficiency and the utilization rate of the grains, and can further effectively avoid wear, microfracture, macrofracture, and falling off of the grains. The improved micro grinding head can effectively increase a material removal rate during use, reduce roughness of a surface of a processing object, and improve processing quality of the surface of the processing object.

For the bionic microtexture micro grinding head described above, when the plurality of grain groups are arranged according to the bionic pinion microstructure, each grain group is disposed along a circumferential side of the grinding head, each groove is provided with a plurality of bends to make the groove provided in a wavy form, and an orientation of the bends of the groove is disposed perpendicular or intersecting the central axis of the connecting member. The grain groups are arranged according to the bionic pinion microstructure. The grain groups are arranged according to a specified direction and have the bends to extend by a length of the groove, so that the micro grinding head has directional flow guidance and resistance reduction functions. The groove is provided between two adjacent grain groups. The groove arrangement can effectively take away heat generated during processing in grinding processing, and is beneficial to controlling a temperature in a bone grinding process.

For the bionic microtexture micro grinding head described above, in consideration of the appropriateness of the structural arrangement, the groove has a specified width and a specified depth, and the depth of the groove is less than a height of the grains in the grain group.

The grain group is located at a boss, and the width of the groove is less than a width of the boss at which the grain group is located.

For the bionic microtexture micro grinding head described above, an arrangement shape of each grain group is consistent with a shape of the groove, and a bending angle at the bends of the groove is an obtuse angle.

Each grain group is provided with at least two rows of grains, and a distance between two adjacent rows of grains in each grain group is less than the width of the groove.

For the bionic microtexture micro grinding head described above, when the plurality of grain groups are arranged according to the bionic scale structure, the groove is formed between two adjacent grain groups, each grain group is sector-shaped, a longer side of the grain group is disposed facing away from the connecting member, and the grain group is arranged to radiate out with a position close to a center of the connecting member as a focus. The grain groups are arranged according to the bionic scale structure. The grain group is arranged to radiate out with a position close to a center of the connecting member as a focus. A comb frame structure is presented on the surface, and a plurality of stripe channels are formed on a surface of the micro grinding head, thereby effectively reducing friction with a coolant, enhancing hydrophobicity, and implementing quick heat exchange.

For the bionic microtexture micro grinding head described above, each grain group includes 6 to 10 grains, all the grains in each grain group are disposed in a plurality of rows, grains in two adjacent rows are staggered, a distance between two adjacent grains in each column of grains at two rows spaced apart by one intermediate row is greater than a distance between two adjacent grains in two adjacent rows of grains, and the distance between two adjacent grains in two adjacent rows of grains is greater than a width of the groove, which helps to further ensure the appropriateness of the arrangement of the grain groups.

For the bionic microtexture micro grinding head described above, when the plurality of grains are arranged according to the bionic phyllotaxis microstructure, this helps to ensure the density of the grains, and is conducive to successful grinding processing. A distance between two adjacent grains in two adjacent rows of grains in an X direction is greater than a distance between the two grains in a Y direction, and the distance between two adjacent grains in two adjacent rows of grains in the X direction is greater than a distance between two adjacent grains in a same row in the X direction.

According to a second aspect, the present invention further provides a processing process of a bionic microtexture micro grinding head, including the following content:
placing a micro grinding head base in a degreasing solution to perform degreasing treatment;
performing structural processing on the micro grinding head base with grain groups arranged according to a bionic pinion microstructure to manufacture a groove in a surface of the micro grinding head base;
pre-plating the micro grinding head base;
performing glue dispensing on the surface of the pre-plated micro grinding head base, and forming glue points on the surface of the micro grinding head base, where the glue points are arranged according to the bionic pinion microstructure or a bionic scale structure or a bionic phyllotaxis microstructure;
performing acid cleaning and oxidation treatment on grains to keep the grains from falling off from the micro grinding head base; and
sprinkling the grains with the oxidation treatment completed on the surface of the micro grinding head base, fastening the grains at the glue points to form a micro grinding head, and performing thick plating on the formed micro grinding head to obtain the bionic microtexture micro grinding head.

For the processing process of the bionic microtexture micro grinding head described above, the degreasing solution contains sodium hydroxide NaOH, sodium carbonate Na₂CO₃, sodium phosphate Na₃PO₄, and sodium silicate Na₂SiO₃, and after the degreasing treatment is performed on the micro grinding head base, roughing treatment is performed on the micro grinding head base, so that micro pits are formed in the surface of the surface of the micro grinding head base, thereby enhancing an adhesive force of a coating.

For the processing process of the bionic microtexture micro grinding head described above, the pre-plating the micro grinding head base includes the following content: placing the micro grinding head base in an electroplating solution that contains nickel sulfate NiSO₄, nickel chloride NiCl₂, boric acid H₃BO₃, and sodium dodecyl sulfate C₁₂H₂₅—OSO₃Na.

The beneficial effects of the present invention above are as follows:

(1) For the arrangement of the micro grinding head in the present invention, the grain groups or the grains are disposed on the surface of the grinding head. The grain groups or the grains are arranged according to various bionic structures, thereby avoiding agglomeration of the grains during electroplating. The layout of grains of a conventional grinding head is changed, and the grains are arranged in order, so that concentration of a grinding force is reduced, and thermal-mechanical damage in a bone grinding process is reduced, thereby avoiding a surgical problem caused by a high temperature. The improved micro grinding head can effectively improve the grinding efficiency and the utilization rate of the grains.

(2) The grain groups and the grains are appropriately arranged on the surface of the grinding head of the micro grinding head in the present invention, and the distribution of the grain groups and the grains is appropriate, so that wear, microfracture, macrofracture, and falling off of the grains can be further effectively avoided. The improved micro grinding head can effectively increase a material removal rate during use, reduce roughness of a surface of a processing object, and improve processing quality of the surface of the processing object.

(3) The grain groups are arranged according to the bionic pinion microstructure in the present invention. The grain groups are arranged according to a specified direction and have the bends to extend by a length of the groove, so that the micro grinding head has directional flow guidance and resistance reduction functions, to effectively make the coolant to overcome the hindrance of an air layer to enter a grinding region. The coolant enters the grinding region to perform effective lubrication and cooling, thereby further reducing a grinding temperature. The groove is provided between two adjacent grain groups. The groove arrangement can effectively take away heat generated during processing in grinding processing, and is beneficial to controlling a temperature in a bone grinding process.

In addition, because debris generated by grinding and particles generated from breakage of the grains gather in the groove in the surface of the grinding head, the pinion-shaped groove helps to guide uniform flow and take away the debris, thereby reducing clogging of the grinding head, reducing a temperature rise caused by the clogging, and improving processing quality of the processing object.

(4) The grain groups are arranged according to the bionic scale structure in the present invention. The grain group is arranged to radiate out with a position close to a center of the connecting member as a focus. A comb frame structure is presented on the surface, and a plurality of stripe channels are formed on a surface of the micro grinding head, thereby effectively reducing friction with a coolant, enhancing hydrophobicity, and implementing quick heat exchange.

(5) In the present invention, the plurality of grains is arranged according to the bionic phyllotaxis microstructure, and the density of the grains is effectively ensured by limiting the distances between two adjacent grains in the X direction and the Y direction.

(6) In the processing process proposed in the present invention, the micro grinding head base is first treated, the groove is processed after the treatment is completed, pre-plating is performed, the pre-plating treatment can enhance stability of subsequent grains, then the grains are treated, and finally the grains are fastened to the micro grinding head base to form the bionic microtexture micro grinding head. An overall process is appropriately set, and acid cleaning and oxidation treatment are performed on the grains, so that the grains can be effectively kept from falling off the micro grinding head base, thereby ensuring the service life of the micro grinding head as a whole.

### BRIEF DESCRIPTION OF THE DRAWINGS

The accompanying drawings of the specification, which constitute a part of the present invention, are used to provide further understanding of the present invention. Exemplary embodiments of the present invention and descriptions thereof are used to explain the present invention, and do not constitute an improper limitation to the present invention.

FIG. 1 is a front view of a bionic microtexture micro grinding head according to Embodiment 1 of the present invention.

FIG. 2 is an enlarged view of a position A in FIG. 1.

FIG. 3 is an enlarged view of a position B in FIG. 1.

FIG. 4 is a front view of a bionic microtexture micro grinding head according to Embodiment 2 of the present invention.

FIG. 5 is an enlarged view of a part in FIG. 4.

FIG. 6 is a front view of a bionic microtexture micro grinding head according to Embodiment 3 of the present invention.

FIG. 7 is an enlarged view of a position C in FIG. 6.

FIG. 8 is a flowchart of a processing process of a bionic microtexture micro grinding head according to Embodiment 4 of the present invention.

### DETAILED DESCRIPTION

It should be noted that the following detailed descriptions are exemplary and are intended to provide further description of the present invention. Unless otherwise specified, all technical and scientific terms used in the present invention have the same meanings as commonly understood by a person of ordinary skill in the art to which the present invention belongs.

It should be noted that the terms used herein are merely intended to describe specific implementations, but are not intended to limit exemplary implementations according to the present invention. As used herein, the singular form is intended to include the plural form, unless the present invention clearly indicates otherwise. In addition, it should be further understood that terms “include” and/or “including” used in this specification indicate that there are features, steps, operations, devices, assemblies, and/or combinations thereof.

In view of the problem in the prior art that thermal damage occurs easily in a bone grinding process as described in the BACKGROUND, to resolve the foregoing technical problem, the present invention provides a bionic microtexture micro grinding head.

### Embodiment 1

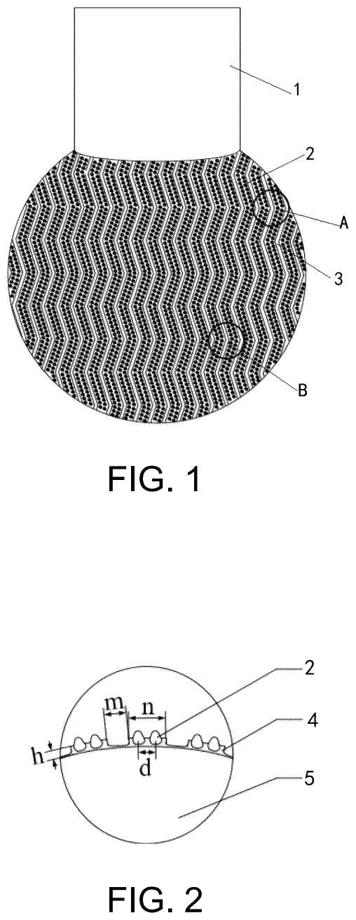

In a typical implementation of the present invention, referring to FIG. 1, a bionic microtexture micro grinding head includes a micro grinding head base. The micro grinding head base includes a connecting member 1. The connecting member 1 is connected to a grinding head 5. The connecting member 1 is specifically a connecting rod. The grinding head 5 is specifically spherical. A notch is provided at an end of the grinding head for a connection to an end of the connecting member. A plurality of grain groups is sequentially disposed on a surface of the grinding head 5. The plurality of grain groups are arranged according to a bionic pinion microstructure.

When the plurality of grain groups are arranged according to the bionic pinion microstructure, each grain group includes a plurality of grains, and when the plurality of grain groups are arranged according to the bionic pinion microstructure, a groove is provided between two adjacent grain groups.

When the plurality of grain groups are arranged according to the bionic pinion microstructure, each grain group is disposed along a circumferential side of the grinding head, each groove is provided with a plurality of bends, orientations of adjacent bends are opposite to make the groove provided in a wavy form, and an orientation of the bends of the groove is disposed perpendicular or intersecting the central axis of the connecting member. The groove extends from one side close to the connecting member to the other end of the grinding head, and the grain groups are arranged according to the bionic pinion microstructure. The grain groups are arranged according to a specified direction and are provided with the bends.

In consideration of the appropriateness of the structural arrangement, the groove 3 has a specified width and a specified depth. In the present embodiment, a depth h (10 μm to 50 μm) of the groove 3 is less than a height of the grains 2 in the grain groups. The grain group is located at a boss. Referring to FIG. 2, a width m (20 μm to 100 μm) of the groove is less than a width n (50 μm to 300 μm) of a boss 4 where the grain groups are located.

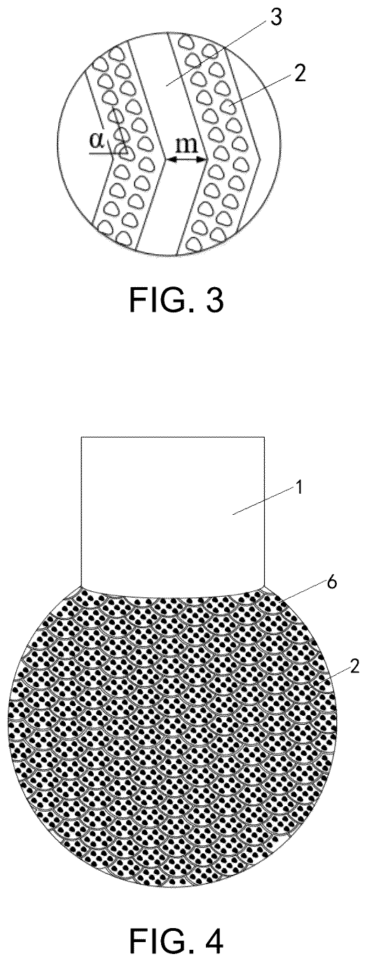

An arrangement shape of each grain group is consistent with a shape of the groove, and a bending angle at the bends of the groove is an obtuse angle. Each grain group is provided with at least two rows of grains. Referring to FIG. 3, an angle α (30° to 75°) between a row in which the grains are located and a horizontal line on a side is an acute angle. In the present embodiment, two rows of grains are provided, grains in two adjacent rows of grains are staggered, and a distance d (20 μm to 50 μm) between two adjacent rows of grains in each grain group is less than the width m (20 μm to 100 μm) of the groove.

It may be understood that, when the micro grinding head is used to perform a bone grinding operation, due to high-speed rotation of the micro grinding head, an air layer is formed on the surface of the grinding head, which hinders a coolant from entering a grinding region. In addition, because the grinding head is in close contact with a workpiece during grinding, the coolant cannot effectively take heat away from the grinding region. In addition, it is difficult for debris generated from grinding to be discharged from the grinding region, and debris gradually accumulates in gaps between the grains, resulting in clogging of the grinding head, affecting processing quality, resulting in a decrease in heat dissipation performance of the grinding head, causing thermal damage to bone tissue, and reducing processing efficiency. In the present embodiment, the plurality of grain groups are arranged according to the bionic pinion microstructure, and flow guidance and resistance reduction effects of the pinion-shaped structure can effectively enable the coolant to overcome the hindrance of an air layer to enter the grinding region. The coolant enters the grinding region to perform effective lubrication and cooling, thereby further reducing a grinding temperature. In addition, because debris generated by grinding and particles generated from breakage of the grains gather in the groove in the surface of the grinding head, the pinion-shaped groove helps to guide uniform flow and take away the debris, thereby reducing clogging of the micro grinding head, reducing a temperature rise caused by the clogging, and improving processing quality of the workpiece.

### Embodiment 2

A difference between the present embodiment and Embodiment 1 lies in that

The plurality of grain groups are arranged according to a bionic scale structure, and are arranged according to a required mesh count (100 # to 1200 #). Referring to FIG. 4, when the plurality of grain groups are arranged according to the bionic scale structure, the groove 3 is formed between two adjacent grain groups 6, and each grain group is sector-shaped. The groove is provided on a circumferential side of the grain group, and each side of the grain group is arc-shaped. In other words, the surface of the grinding head is covered by scales formed by the grain groups, and thicknesses of all the grain groups are kept the same or close. A longer side of the grain group is disposed facing away from the connecting member. The grain groups are arranged according to the bionic scale structure. The grain group is arranged to radiate out with a position close to a center of the connecting member as a focus. A comb frame structure is presented on the surface. Center lines (along center points of the sector-shaped grain groups) of some grain groups are located on a same straight line, and the straight line is disposed parallel to or intersecting the central axis of the connecting member. When intersecting, an angle between the straight line and the central axis of the connecting member ranges from 0° to 10°. A plurality of stripe channels are formed on a surface of the micro grinding head, thereby effectively reducing friction with a coolant, enhancing hydrophobicity, and implementing quick heat exchange.

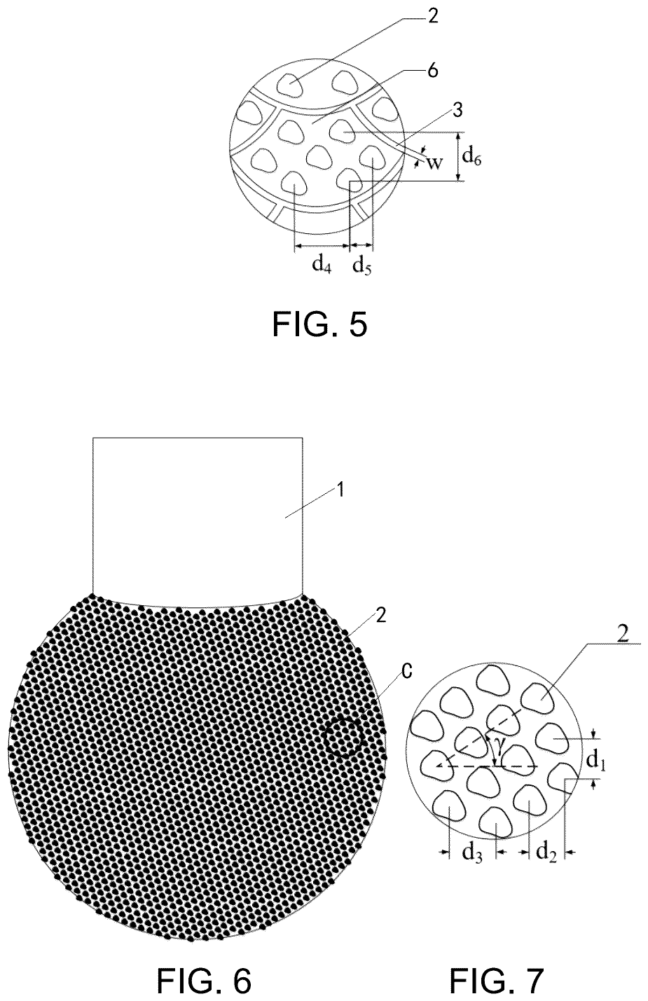

Each grain group includes 6 to 10 grains, and specifically, 7 grains. All the grains in each grain group are disposed in a plurality of rows. In the grain group, two grains are disposed in a row close to the connecting member, three grains are disposed in the middle, and two grains are disposed in a row far away from the connecting member. Grains in two adjacent rows are staggered. Referring to FIG. 5, a distance d₄ (100 μm to 150 μm) between two adjacent grains in each row in a horizontal direction of the grains is greater than a distance d₆ (90 μm to 120 μm) between two adjacent grains in each column of grains at two rows spaced apart by one intermediate row. The distance d₆ (90 μm to 120 μm) between two adjacent grains in each column of grains at two rows spaced apart by one intermediate row is greater than a distance d₅ (60 μm to 100 μm) between two adjacent grains in two adjacent rows of grains. The distance d₅ (60 μm to 100 μm) between two adjacent grains in two adjacent rows of grains is greater than a width w (20 μm to 100 μm) of the groove, which helps to further ensure the appropriateness of the arrangement of the grain groups.

It should be noted that, because a distance between two adjacent grain groups in the present embodiment is small, small parameters are used when a laser beam is used to perform structural processing on the surface of the micro grinding head base. For example, in the present embodiment, processing may be performed by using parameters being a current intensity of 20 A, a pulse interval of 20 ns, a laser frequency of 20 kHz, and a marking speed of 20 mm/s, to ensure the size and precision of the groove.

For the micro grinding head in Embodiment 1 or Embodiment 2, when the micro grinding head grinds bone tissue, wear of grains is inevitable. How to change a grain structure to ensure the wear resistance of the micro grinding head is an important indicator for evaluating the performance of the micro grinding head, and a grinding ratio G is usually used to determine the wear resistance of the micro grinding head, and indicates a ratio of an amount of material removed from a processing object to a wear amount of the micro grinding head within a unit time. A higher grinding ratio indicates better wear resistance and a longer service life, and a calculation formula of the grinding ratio G is as follows:

$$G = \frac{{V}_{m}}{{V}_{t}} .$$

In the formula, Vₜ is the wear amount of the micro grinding head, Vₘ is a removed volume of the processing object. For planar grinding, a calculation formula of the grinding ratio G is as follows:

$$G = \frac{Bld}{2 π r W Δ r} .$$

In the formula, l is the length of a workpiece, B is a grinding width, r is the radius of the grinding head, W is the width of the grains, d is a grinding depth, and Δr is a wear amount of a radius of the grinding head within a unit time. The grinding ratio G can be changed by adjusting the foregoing parameters.

It should be noted that the presence of a rough surface makes an actual solid-liquid contact area larger than an apparent geometric contact area, which enhances hydrophilicity (or hydrophobicity) in geometry. Assuming that coolant drops always fill up the grooves in the surface of the grinding head, a relationship between an apparent contact angle β* of the rough surface and βₑ is:

$$cos {β}^{*} = r ( {γ}_{S G} - {γ}_{S L} ) / {γ}_{L G} = r cos {β}_{e} .$$

In the formula, γ_{SG}, γ_{SL}, and γ_{LG} are respectively surface tensions at solid-gas, solid-liquid, and liquid-gas contact surfaces; and r is a roughness factor of a material surface, representing a ratio of an actual contact area to an apparent contact area, and r≥1. Therefore, the apparent contact angle can be regulated by changing surface roughness of the grinding head, thereby changing wettability of the surface of the grinding head.

In addition, contact between coolant drops and the rough surface is a composite contact. The drops cannot fill up the grooves in the rough surface of the grinding head, and trapped air remains under drops in the grooves. Therefore, apparent liquid-solid contact is actually formed by liquid-solid and gas-solid contact. From a thermodynamic perspective:

$$d G = {f}_{s} ( {γ}_{S L} - {γ}_{S G} ) d x + ( 1 - {f}_{s} ) {γ}_{S G} d x + {γ}_{L G} d x cos {β}^{*} .$$

At drop balance, the apparent contact angle β* of the rough surface is an average value of an intrinsic contact angle βₑ of a smooth and flat surface and 180°:

$$cos {β}^{*} = {f}_{s} ( 1 + cos {β}_{e} ) - 1 .$$

In the formula, fₛ is an area fraction (fₛ<1) of protruding solids in a composite contact surface.

The most important factor that affects the dynamic behavior of drops on a surface is a three-phase contact boundary. As shown in FIG. 5, at drop balance, the contact angle is β (a state d); if a small amount of liquid is added and the solid-liquid-gas three-phase contact boundary remains unchanged, the contact angle definitely increases to β₂ (a state e). In contrast, if a small amount of liquid is extracted and at the same time the solid-liquid-gas three-phase contact boundary is kept unchanged, the contact angle definitely decreases to β₁ (a state c). Assuming that the solid-liquid-gas three-phase contact boundary has only three interface tensions, at balance, states d, e, and c all have:

$$cos β = cos {β}_{1} = cos {β}_{2} - \frac{{γ}_{S G} - {γ}_{S L}}{{γ}_{L G}} .$$

The above shows that a drop at a balanced spread position needs to overcome a pinning effect of a solid on a contact boundary to continue to spread along a wall surface of the solid. In a cooling process for skull grinding in neurosurgery, a coolant continuously enters a grinding region. A previous coolant drop impacts a bone surface at a specified speed and a specified angle to spread into a liquid film. The most favorable state for cooling and lubrication effects is that a subsequent drop impacts the position of the previous drop to continue to spread, i.e., a coolant drop can overcome a pinning effect of the rough bone surface on a contact boundary of the drop.

### Embodiment 3

A difference between the present embodiment and Embodiment 1 lies in that:

Referring to FIG. 6, the plurality of grains is arranged according to a bionic phyllotaxis microstructure. when the plurality of grains are arranged according to the bionic phyllotaxis microstructure, the plurality of grains are disposed in a plurality of rows and a plurality of columns, rows in which some grains are located and columns in which some grains are located are respectively disposed obliquely with respect to a center-crossing radial plane of the grinding head, an angle between each row of grains and a horizontal line is γ and is less than 45°, and the center-crossing radial plane is disposed perpendicular to a central axis of the connecting member.

When the plurality of grains are arranged according to the bionic phyllotaxis microstructure, this helps to ensure the arrangement density of the grains at the grinding head, and is conducive to successful grinding processing. A distance between two adjacent grains in two adjacent rows of grains in an X direction is greater than a distance between the two grains in a Y direction. Referring to FIG. 7, a distance d₃ between two adjacent grains in two adjacent rows of grains in the X direction is greater than a distance d₂ between two adjacent grains in a same row in the X direction. The distance d₃ between two adjacent grains in two adjacent rows of grains in the X direction is greater than a distance d₁ between two adjacent grains in two adjacent rows of grains in the Y direction.

### Embodiment 4

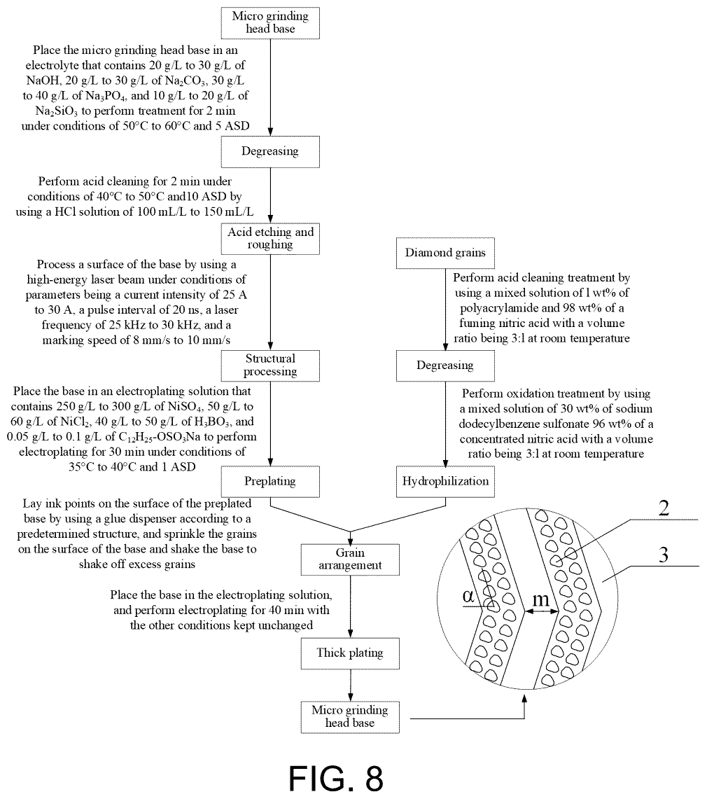

The present embodiment discloses a processing process of the bionic microtexture micro grinding head in Embodiment 1 or Embodiment 2 or Embodiment 3. Referring to FIG. 8, the processing process includes the following content:

Step 1: Place a micro grinding head base in an electrolyte that contains 30 g/L of sodium hydroxide NaOH, 30 g/L of sodium carbonate Na₂CO₃, 40 g/L of sodium phosphate Na₃PO₄, and 20 g/L of sodium silicate Na₂SiO₃, perform treatment for 2 min under conditions of 50° C. and 4 ASD (amperes per square decimeter), perform drying, and remove oil stain, solid particles, and other impurities on a surface of the micro grinding head base; and then place the base in 150 mL/L of a hydrogen chloride HCl solution to perform roughing treatment, perform acid cleaning for 2 min under conditions of 40° C. and 9 ASD, perform drying, and forming micro pits in the surface of the micro grinding head base, to enhance an adhesive force of a coating.

Because an oxide layer exists on the surface of the untreated micro grinding head base, if electroplating is directly performed, a bonding force between the coating and the surface of the micro grinding head base is excessively low. As a result, the coating falls off from the surface of the micro grinding head base, processing quality is affected, and the service life of the micro grinding head is reduced.

Step 2: Cut the surface of the micro grinding head base according to a specified pattern by using a laser beam, and perform evaporation, melting, and other processes to manufacture bionic pattern grooves with a specified depth in the surface of the base.

Specific parameter ranges are a current intensity of 25 A, a pulse interval of 20 ns, a laser frequency of 30 kHz, and a marking speed of 8 mm/s.

Step 3: Perform pre-plating treatment on the micro grinding head base. Before the grains are fastened on the surface of the micro grinding head base, the pre-plating treatment needs to be performed on the micro grinding head base to enhance the stability of the grains. The micro grinding head base is placed in an electroplating solution that contains 250 g/L of nickel sulfate NiSO₄, 50 g/L of nickel chloride NiCl₂, 40 g/L of boric acid H₃BO₃, and 0.05 g/L of sodium dodecyl sulfate C₁₂H₂₅—OSO₃Na, electroplating is performed for 30 min under conditions of 40° C. and 2 ASD, and drying treatment is not performed after the electroplating is completed.

Step 4: Perform glue dispensing on the surface of the pre-plated micro grinding head base, and forming glue points on the surface of the micro grinding head base, where the glue points are arranged according to the bionic pinion microstructure or a bionic scale structure or a bionic phyllotaxis microstructure.

Step 5: Perform hydrophilization treatment on diamond grains. To remove oil stain, solid particles, and other impurities on a surface of the diamond grains, the grains are placed in a mixed solution of 1 wt % of polyacrylamide and 98 wt % of a fuming nitric acid with a volume ratio being 3:1 to perform acid cleaning treatment at room temperature, and after the acid cleaning treatment is completed, the grains are rinsed with deionized water three times. A mixed solution of 30 wt % of sodium dodecylbenzenesulphonate and 96 wt % of a concentrated nitric acid with a volume ratio being 3:1 is used to perform oxidation treatment on the diamond grains at room temperature, and the diamond grains are washed with deionized water, until a pH value is kept within a range of 6.5 to 7.0.

Step 6: Perform thick plating on the micro grinding head. The diamond grains with the hydrophilization completed are uniformly sprinkled on the surface of the base. The grains are fastened to the glue points. Because drying treatment is not performed on the base after the pre-plating, the diamond grains may be adhered to a residual electroplating solution on the surface of the micro grinding head base, and therefore a fastening operation does not need to be performed on the grains. The components of the electroplating solution are kept unchanged, electroplating is performed for 1.5 min under conditions of 1 ASD and 40° C., and the operation is repeated 8 times. After the operations are completed, the other conditions are kept unchanged, and electroplating is performed for 40 min to perform thick plating.

It should be noted that an unsmooth surface may appear on a coating in an electroplating process mainly due to two reasons: the first reason is that oil stain, solid particles, and other impurities exist on the surfaces of the micro grinding head base and the grains without degreasing treatment, and the second reason is a hydrogen evolution reaction in the electroplating process.

When degreasing treatment is not performed on the micro grinding head base and the grains, impurities attached to the surfaces thereof contaminate the electroplating solution during electroplating, resulting in an insufficient purity of the coating, and forming pits in the surface of the coating. Under this reason, the pits are generated mainly due to two reasons: The first reason is that various insoluble or undissolved solid particulates exist in the electroplating solution or the surfaces of the untreated diamond grains and are referred to as mechanical impurities. When the solid particulates sink at the surface of the micro grinding head base and are covered in the coating, because the solid particulates are larger than suspended solid particulates, the coating is clearly rough. Therefore, oil stain and impurities can be effectively removed by performing degreasing treatment on the micro grinding head base and performing hydrophilization treatment on the grains, making the surface of the coating smooth.

In addition, the hydrogen evolution reaction occurs in the electroplating process mainly due to water decomposition in the electrolyte and anion reduction in the electrolyte. In an electrolyzer, an electrode transfers a current to the base, and water molecules in the electrolyte are decomposed into hydrogen and oxygen. In this water decomposition reaction, some anions are reductive, i.e., the release of hydrogen under the action of a current in an electroplating process is inevitable, and is especially common in direct-current electroplating. The hydrogen evolution in electroplating is mainly because hydrogen generated in the water decomposition reaction generates bubbles on a metal surface.

The electroplating process of the micro grinding head base may include two parts: In the first part, as a current density in polarization at a cathode increases rapidly, Ni⁺ in the electrolyte acquire electrons and is reduced into nickel to be deposited onto a surface of the base. In the second part, as the polarization continues, an oxidation-reduction potential for H⁺ in the electrolyte is reached, a large amount of hydrogen H₂ is evolved, i.e., the hydrogen evolution reaction occurs. In addition, it is found by observing the surface of the micro grinding head base before and after electroplating that the coating is not uniform. As can be seen through analysis, a process of acid etching and roughing is performed first on the micro grinding head base before electroplating, to form pits of different sizes in the surface of the grinding head. Convex points represent higher skewness and kurtosis, and can promote nucleation and growth of the coating, and height differences between the convex points and concave points lead to higher surface roughness. In another aspect, due to a leveling effect, for changes in the thickness of the coating, the pits are filled up first, to keep the final uniformity and surface roughness.

In the processing process proposed in the present embodiment, the micro grinding head base is first treated, the groove is processed after the treatment is completed, pre-plating is performed, the pre-plating treatment can enhance stability of subsequent grains, then the grains are treated, and finally the grains are fastened to the micro grinding head base to form the bionic microtexture micro grinding head. An overall process is appropriately set, and acid cleaning and oxidation treatment are performed on the grains, so that the grains can be effectively kept from falling off the micro grinding head base, and the electroplating process can effectively fill up pits in a surface of an object, which is conducive to the uniformity of the surface of the eventually formed micro grinding head, thereby ensuring the service life of the micro grinding head as a whole.

The foregoing descriptions are merely preferred embodiments of the present invention, but are not intended to limit the present invention. A person skilled in the art may make various modifications and changes may the present invention. Any modification, equivalent replacement, improvement, or the like made within the spirit and principle of the present invention shall fall within the protection scope of the present invention.

What is claimed is:

1. A bionic microtexture micro grinding head, comprising a micro grinding head base, wherein the micro grinding head base comprises a connecting member, the connecting member is connected to a grinding head, a plurality of grain groups or a plurality of grains are sequentially disposed on a surface of the grinding head, the plurality of grain groups are arranged according to a bionic pinion microstructure or a bionic scale structure, and the plurality of grains are arranged according to a bionic phyllotaxis microstructure;
when the plurality of grain groups are arranged according to the bionic pinion microstructure or the bionic scale structure, each of the grain groups comprises a plurality of grains, and each of grooves is provided between two adjacent grain groups of the grain groups;
when the plurality of grains are arranged according to the bionic phyllotaxis microstructure, the plurality of grains are disposed in a plurality of rows and a plurality of columns, some of the rows in which some of the grains are located and some of the columns in which some of the grains are located are respectively disposed obliquely with respect to a center-crossing radial plane of the grinding head, and the center-crossing radial plane is disposed perpendicular to a central axis of the connecting member; and
the grains are disposed protruding from the surface of the grinding head.

2. The bionic microtexture micro grinding head according to claim 1, wherein when the plurality of grain groups are arranged according to the bionic pinion microstructure, each of the grain groups is disposed along a circumferential side of the grinding head, each of the grooves is provided with a plurality of bends to make the groove provided in a wavy form, and an orientation of the bends of the groove is disposed perpendicular or intersecting the central axis of the connecting member.

3. The bionic microtexture micro grinding head according to claim 2, wherein the groove has a width and a depth, and the depth of the groove is less than a height of the grains in the grain group; and
the grain group is located at a boss, and the width of the groove is less than a width of the boss at which the grain group is located.

4. The bionic microtexture micro grinding head according to claim 3, wherein an arrangement shape of each of the grain groups is consistent with a shape of the groove, and a bending angle at the bends of the groove is an obtuse angle; and
the grains in each of the grain groups are arranged in at least two rows, and a distance between the grains in two adjacent rows of the at least two rows in each of the grain groups is less than the width of the groove.

5. The bionic microtexture micro grinding head according to claim 1, wherein when the plurality of grain groups are arranged according to the bionic scale structure, each of the grain groups is sector-shaped, the grain group is arranged to radiate out with a position close to a center of the connecting member as a focus, and a longer side of the grain group is disposed facing away from the connecting member.

6. The bionic microtexture micro grinding head according to claim 5, wherein the grains in each of the grain groups comprises 6 to 10 grains, all the grains in each of the grain groups are arranged in a plurality of rows, grains in two adjacent rows of the rows are staggered, a distance between two adjacent grains in each of columns at two rows of the rows spaced apart by one intermediate row of the rows is greater than a distance between two adjacent grains of the grains in the two adjacent rows of the rows, and the distance between two adjacent grains of the grains in the two adjacent rows of the rows is greater than a width of the groove.

7. The bionic microtexture micro grinding head according to claim 1, wherein when the plurality of grains are arranged according to the bionic phyllotaxis microstructure, a distance between two adjacent grains in two adjacent rows of the rows in an X direction is greater than a distance between the two grains in a Y direction, and the distance between two adjacent grains in two adjacent rows of the rows in the X direction is greater than a distance between two adjacent grains in a same row of the rows in the X direction.

8. A processing process of the bionic microtexture micro grinding head according to claim 1, comprising:
placing a micro grinding head base in a degreasing solution to perform degreasing treatment;
performing structural processing on the micro grinding head base with grain groups arranged according to a bionic pinion microstructure to manufacture a groove in a surface of the micro grinding head base;
pre-plating the micro grinding head base;
performing glue dispensing on the surface of the micro grinding head base which is pre-plated, and forming glue points on the surface of the micro grinding head base, wherein the glue points are arranged according to the bionic pinion microstructure or a bionic scale structure or a bionic phyllotaxis microstructure;
performing acid cleaning and oxidation treatment on grains to keep the grains from falling off from the micro grinding head base; and
sprinkling the grains with the oxidation treatment completed on the surface of the micro grinding head base, fastening the grains at the glue points to form a micro grinding head, and performing thick plating on the micro grinding head which is formed to obtain the bionic microtexture micro grinding head.

9. The processing process of the bionic microtexture micro grinding head according to claim 8, wherein the degreasing solution contains sodium hydroxide, sodium carbonate, sodium phosphate, and sodium silicate, and after the degreasing treatment is performed on the micro grinding head base, roughing treatment is performed on the micro grinding head base.

10. The processing process of the bionic microtexture micro grinding head according to claim 8, wherein the pre-plating the micro grinding head base comprises: placing the micro grinding head base in an electroplating solution that contains nickel sulfate, nickel chloride, boric acid, and sodium dodecyl sulfate.
